(19) United States
(12) Patent Application Publication
(10) Pub. No.: US 2012/0231492 A1
(43) Pub. Date: Sep. 13, 2012

(54) SENSOR FOR DETECTING MICROORGANISMS AND CORRESPONDING PROCESS

(76) Inventors: Steve Bitterly, Agoura Hills, CA (US); Jack Bitterly, Woodland Hills, CA (US); Jean Bitterly, (US)

(21) Appl. No.: 13/415,702

(22) Filed: Mar. 8, 2012

Related U.S. Application Data
(60) Provisional application No. 61/450,342, filed on Mar. 8, 2011.

(51) Int. Cl. G01N 27/00; A61B 5/145; C12M 1/34

(52) U.S. Cl. 435/34; 435/287.1; 600/345

(57) ABSTRACT

Microbial fuel cells generate an electrical signal when microbes enter the cells through a semipermeable membrane. By reading and analyzing the signal from one or more such fuel cells can indicate infection in people or animals, indicate pathogens growing in food or show mold growth. Insofar as different microbes have specific metabolisms, the signal may be used to determine which microbe is present.

### RELATED APPLICATION

This application claims priority to provisional application No. 61/450,342, filed Mar. 8, 2011, by Jack G. Bitterly and Steve E. Bitterly.

### BACKGROUND

1. Field

A sensor and corresponding process detects the presence of microorganisms such as bacteria, molds or viruses and identifies the microorganism.

2. General Background and State of the Art

Identifying whether a particular pathogenic microorganism is present can be crucial for human or animal patients and for other applications.

One way to determine the presence of bacteria on or in a patient is through culturing a sample. Though techniques for aerobic and anaerobic bacteria are different, they are well known. If a physician suspects, for example, that a child has strep throat, a Group A streptococci infection, the physician or nurse takes a mucus sample from the child's throat with a swab and rubs the swab onto agar in a Petri dish. There, any bacteria, if present, incubate. After several days, a technician can determine if streptococci is present. One can also culture microorganism from different sites such as around a wound or surgery or in the blood. Similar incubation techniques also work for molds although the culturing techniques may differ. Some bacteria and many molds also resist culturing. Nevertheless, culturing on a medium in a Petri dish is an accepted practice.

This culturing procedure takes time and is expensive. Physicians and veterinarians usually want to know immediately whether to begin an antibiotic regimen. They face pressure to prescribe antibiotics if they suspect a bacterial infection. Standard protocol following some surgeries requires administering antibiotics even without symptoms of an infection. However, many believe that overuse of antibiotics is a serious problem that has caused antibiotic resistant strains by many bacteria. Thus, a physician confident that the patient or wound is free from infection might be able to avoid using antibiotics. If infection has begun, however, the physician can be confident that the patient needs antibiotics.

Bacteria multiply rapidly, exponentially under ideal conditions. When relatively few bacteria are present, the patient may exhibit no symptoms of an infection, but culturing samples may be hit or miss. A sample from part of a wound may yield bacteria; another part of the wound may yield none.

Delay seems to be built into culturing. If the physician does not begin treatment until he or she receives positive results, the infection may become serious. Similarly, one sample may find one strain of bacteria, but may overlook a more serious, virulent pathogen. Antibiotics administered for the bacteria found in the sample may not be ideal for other strains in adjacent areas. Delaying treatment may allow an infection to grow and cause severe illness or death.

Hospital- or office-based physicians rarely perform culturing themselves. In an office practice, the physician sends the sample to an outside laboratory. Thus, the transportation of the sample causes delay. Delay also occurs as the lab performs the tests and then reports to the physician. Even hospitals, which may have their own on-site labs, sample transportation to the lab and delay in transmitting results are usual. For hospitals that subcontract lab services to off-site labs, the delay can increase.

Bacteria also contaminate food. For example, according to the United States Centers for Disease Control (CDC), more than 70,000 Americans are infected annually by E coli O157:H7, usually from contaminated ground beef. More than 60 of the infected die, and others suffer severe symptoms. People also have been sickened or died from eating infected milk, cheese and chicken. The United States government estimates that food-borne diseases cause more than 80 million illnesses, 350,000 hospitalizations, 5,000 deaths, and US$3.1 billion in costs yearly. Meat spoilage due to bacterial contamination, a main cause of food-borne diseases, also results in US$65 billion in product losses annually to retailers and consumers. Detecting such bacteria is difficult, especially for the consumer. A package of ground beef may be acceptable at a supermarket, but the bacteria may reproduce rapidly after it is purchased.

Though food processors test for the presence of bacteria—some people complain that the testing is inadequate—food may leave a processor with undetectable bacteria levels. The bacteria reproduce rapidly if the food is transported at a warm temperature, kept on a warm loading dock at a market or restaurant, kept in a consumer's trunk while driving home, maintained too long in a refrigerator or allowed to warm to room temperature before cooking. Unfortunately, meat contaminated with E. coli usually looks and smells normal. The United States government warns consumers and restaurants to cook ground beef to 160° F. (71° C.), the temperature that kills that E. coli.

Most food safety experts concentrate on the following nine principal bacteria:

Campylobacter jejuni is found in intestinal tracts of animals and birds, raw milk, untreated water and sewage sludge. Most transmission is from contaminated water, raw milk, and raw or under-cooked meat, poultry or shellfish. Campylobacter jejuni is a gram-negative bacterium like bacilli. It uses oxygen as its final electron acceptor.

Clostridium botulinum is widely distributed in nature including soil and water, on plants and in intestinal tracts of animals and fish. It grows only in little or no oxygen. Transmission usually is from improperly canned foods, garlic in oil and vacuum-packaged and tightly wrapped food. Infection can be fatal in three to ten days if not treated. Clostridium botulinum is a gram-positive bacterium that is typically rod-shaped and arranged as singles, pairs, or chains. The spores grow under favorable conditions (anaerobiosis and substrate-rich environments) and begin to produce toxins as they rapidly propagate.

Clostridium perfringens is found in soil, dust, sewage and animal and human intestinal tracts. It grows only in little or no oxygen. It is called “the cafeteria germ” because many outbreaks result from food left for long periods in steam tables or at room temperature. Cooking destroys the bacteria, but some toxin-producing spores may survive. Clostridium perfringens are non-motile, rod-shaped, gram-positive bacteria. It is an anaerobic bacterium, which acquires energy by performing anaerobic respiration using nitrate as its electron acceptor.

Escherichia coli O157:H7 is found in the intestinal tracts of some mammals, raw milk and unchlorinated water. It is one of several E. coli strains causing human illness. It usually is transmitted through contaminated water, raw milk, raw or rare ground beef, unpasteurized apple juice or cider and uncooked fruits and vegetables. Person-to-person transmission also can occur. E. coli serotype O157:H7 is a mesophilic, gram-negative rod-shaped (bacilli) bacterium. It can perform complicated metabolism to maintain its cell growth and cell division and possesses operons for transport and utilization of sucrose, urease, and sorbose.

Salmonella (over 1,600 types) can be found in the intestinal tract and feces of animals and in raw eggs. Salmonella transmission comes from raw or undercooked eggs, poultry, seafood and meat and from raw milk and dairy products. Salmonella Enteritidis is a rod-shaped, gram-negative, proteobacteria that is non-motile. These facultative anaerobes are well adapted to survive in conditions with or without oxygen, allowing them to live in diverse environments. S. Enteritidis require glucose to survive and uses mixed acid heterofermentation of glucose to produce energy. This method of metabolism releases carbon dioxide and hydrogen gas as bi-products.

Streptococcus Group A is found in noses, throats, pus, sputum, blood and stools of humans. It is transmitted from people to food from poor hygiene, ill food handlers or improper food handling. Many outbreaks come from raw milk, ice cream, eggs, lobster, salads, custard and pudding allowed to stand at room temperature for several hours between preparation and eating. Streptococcus Group A is a gram-positive bacterium, which is nonmotile and does not form spores. Rather than using aerobic or anaerobic respiration, Group A Streptococcus uses fermentation, a metabolic pathway of lesser efficiency, which makes the cells grow more slowly.

Listeria monocytogenes exist in intestinal tracts of humans and animals, milk, soil, leaf vegetables and processed foods. It can grow slowly at refrigerator temperatures. It is transmitted from soft cheese, raw milk, improperly processed ice cream, raw leafy vegetables, meat and poultry. It causes illness itself instead of from toxins. Listeria monocytogenes are gram-positive rod-shaped bacteria that form single short chains. They do not form spores or branch and are motile via peritrichous flagella at room temperature (20° C.-25° C.).

Shigella (over 30 Types) is found in human intestinal tract. They are rare in other animals. It is transmitted from person to person by a fecal-oral route and by fecal contamination of food and water. Most outbreaks result from food, especially salads, prepared and handled by workers using poor personal hygiene. Shigella is a non-spore-forming, gram-negative bacterium that aids in the facilitation of intracellular pathogens. Shigella pathogens use a mixed acid fermentation pathway to metabolize substrates. Products of this anaerobic pathway include ethanol, acetic acid, lactic acid, succinic acid, formic acid, and carbon dioxide.

Staphylococcus aureus is found on humans (skin, infected cuts, pimples, noses, and throats). Most transmission occurs through improper food handling. It multiplies rapidly at room temperature to produce a toxin that causes illness. Staphylococci are spherical gram-positive bacteria, which are immobile and form grape-like clusters. The central routes of glucose metabolism are the Embden-Meyerhof-Parnas (EMP) pathway and the pentose phosphate cycle. Lactate is the end product of anaerobic glucose metabolism, and acetate and CO₂ are the products of aerobic growth conditions.

Testing for molds is usually very different. Most molds grow in damp, dark regions of buildings or on food. Molds can adversely affect the buildings, e.g., by eating through walls, floors, ceilings and internal supports. Molds also produce spores that may be toxic to people. Pulmonary hemorrhage/hemosiderosis, and aspergillosis are examples of diseases associated with mold exposure.

Molds require spores to reproduce and spread. Ambient indoor and outdoor air contains large quantities of mold spores. When they land on damp soil or vegetation outdoors, they grow. However, the applicants' device and process are concerned primarily with mold growing on damp indoor surfaces and on food, or spores transported through building air ducts. Bread, especially artisan breads made without preservatives, is attacked by molds such as Neurospora crassa. Usually, mold growth is visible, and the offending food discarded. Other food mold is not as easily detected. Aspergillus flavus is a common mold on peanuts and may be present on other legumes and grains. That mold generates the toxin aflatoxin, which is a carcinogenic and which can be deadly if enough is eaten.

Though most food mold is easily detected by looking at the food, a mold growing in buildings may be hidden behind walls, in attics, in ducting and elsewhere. Eliminating all mold and spores in buildings is nearly impossible. Most preventative measures concentrate on eliminating excessive moisture or accumulated water, but a building owner may easily overlook a dripping pipe, water condensing from a cool surface or even a child's water spill on the carpet.

Mold detection usually relies on sight and smell. Many molds smell bad, so people often determine molds' presence by smell. Mold colonies may be visible unless they are behind walls, above ceilings, under flooring or in other inaccessible locations. Occasionally, people resort to monitoring building air especially if the occupants are sensitive to small quantities of building mold.

Air monitoring requires sampling of indoor and outdoor air simultaneously. Because mold spores are everywhere, a high concentration of indoor spores becomes relevant only if the concurrent outdoor sample concentration is lower. However, most experts believe that routine air sampling is not beneficial because visual and scent mold detection usually works. Nevertheless, relying on scent to locate a mold may be difficult. In addition, many molds are difficult to detect through standard tests. Therefore, air sampling can yield false negative results.

Identifying the particular mold usually is not crucial because remediation techniques are similar for most molds. However, identifying the particular bacteria infecting a person or animal usually is important because particular antibiotics target particular bacteria.

Instead of relying on Petri dishes for microorganism detection, one can consider using various electronic devices for microbial detection including the following:

Frey, U.S. Publ. Appl. No. 2004/0014054

Ikeda, U.S. Pat. No. 5,565,085

Stetter, U.S. Publ. Appl. No. 2002/019265.

Thewes, U.S. Publ. Appl. No. 2004/0045839

Yang, U.S. Publ. Appl. No. 2004/0050694

Gelbart, U.S. Publ. Appl. No. 2004/0038420

Huang, U.S. Pat. No. 6,738,734

Andersson, U.S. Publ. Appl. No. 2004/0002167

Glasel, U.S. Publ. Appl. No. 2004/0106162

Bodenhamer, U.S. Publ. Appl. No. 2002/0045200

Conventional biological or microbial fuel cells convert biochemical energy directly into electricity. The driving force of a biological fuel cell is the redox reaction of a carbohydrate substrate such as glucose and methanol using a microorganism or an enzyme as the catalyst. Standard fuel cells rely on noble metals as their catalysts, but the catalysts in biological fuel cells are microorganisms or enzymes. Working conditions in biological fuel cells are mild, i.e., neutral solutions at or near room temperature.

Microbial fuel cells have several, potential uses. Using microbial fuel cells to generate electricity for power is one of the more talked-about applications. Much wastewater contains organic material, which can drive microbial fuel cells. The bacteria would serve two functions, consuming waste for purification purposes and generating electricity. Microbial fuel cells also can act as a sensor to test wastewater for organic materials. Not all bacteria generate the same electrical output in fuel cells. Therefore, microbial fuel cells for power generation typically concentrate on a limited type of bacteria. The fuel cells also are costly, rigid, and semi-permanent. Rather than generating electricity for power, applicants rely on microbes producing measurable signals, or characteristic signals indicating known pathogens under controlled conditions such as temperature and the type of carbohydrate food source.

### SUMMARY

Applicants' sensor uses the metabolic reactions of bacteria or other microbes to produce a quantifiable and measurable voltage signal. An electronic processor, recorder or other device reads and records such signals. The biological sensor is inexpensive and disposable and is useful for detecting the presence of living pathogens or other living substances. The sensor or detector can be made very small (<1 mm² or smaller) to large (>multiple m²), and can be constructed from inexpensive and abundant materials. Information from the device could lead to determining the particular type and kind of pathogen(s) present.

The biosensors may have two “half-cell” reactions that are catalyzed by the metabolic presence of bacteria or other microbes within the sensor. A single-chamber alternative also is contemplated. Inside the sensor, a substrate or carbohydrate material is added that is advantageous to the growth of bacteria or other microbes. The device can use specific carbohydrates if a physician suspects specific microbes.

Applications are numerous including pathogen detection in medicine and surgery, food and food packaging of meats, fish, dairy and other foods, residential and commercial buildings for mold detection or other pathogens (including airborne). Law enforcement, the military, homeland security and emergency responders also have applications. The sensors have other hospital uses apart from direct use with patients. Hospitals are concerned with detecting airborne diseases from air conditioner vents. Hospitals maintain detailed nosocomial rate indexes to measure the rate of infections (from unknown sources) present in their hospital.

Each bacterium has its own specific growth rate under controlled fuel-cell conditions and temperatures. Therefore, each produces its own specific voltage versus time growth-plot. To differentiate between different bacteria, sensors in an array of sensors may have different substrates and different inhibitors. Inhibitors are weak, specific antibiotics that prevent growth of specific bacteria. A semipermeable membrane inside the biosensor, which can transfer hydrogen ions (protons) given off by the metabolizing microorganism, separates the sides of the biosensor. By knowing the voltage versus growth-time versus temperature for the more common bacteria, a system including the sensors may use algorithms to analyze outputs from an array of micro-sensors. The results can be used to narrow unknown bacteria down to one or more probable candidates.

One can use the same or similar techniques to detect molds or other living substances. Although much of this patent applications uses the term “microbe” and deals with bacteria, the discussions are intended to deal with molds too. To detect the growth of molds, the sensor uses substrate materials that are advantageous to mold. In the mold sensor, antibiotics may be added to the substrate to decrease the chance or eliminate false-positive detections of bacteria. Initial building construction or later construction can install these arrays at predetermined locations in homes or commercial buildings. For example, the arrays could be attached at the baseboard of the walls or on floors below carpet padding to detect if mold occurs due to accidental flooding. In these examples, the sensors remain dormant until they are activated by the presence of mold growth.

Determining the presence of sufficient mold spores without determining the species of mold might be sufficient. However, to determine the presence of a specific mold, the present device may use particular substrates and inhibitors to determine which molds are present. By similar methods used for bacteria detection and determination, the device may use an array of sensors. Each sensor is designed for a particular mold species. By analyzing the signals from each sensor, the system determines which mold is present by a process of elimination.

For medical and hospital use, bandages could include an array of disposable sensors. Sensors may be embedded into a patient during or after surgical procedures. These types of embedded sensors may be very small (needle size), which may facilitate their removal after healing or after the critical period of concern. Though applicants discuss physicians and human patients, veterinarians can use similar sensors on animals.

Other applications of the biosensor device and system include inexpensive and disposable sensors for the food industry to determine whether meats, cheeses, other dairy products and other food are beginning to spoil. Integrating a small, inexpensive sensor into food packaging could detect the presence of bacteria and register a tiny voltage. Applying the voltage to voltage sensitive paper could change the paper's color (e.g., green to yellow to red). Green might mean that no bacteria are detected; yellow could mean that some detection of bacteria is present, and red could signal that the food is spoiled. Such a sensor could save food from early disposal after the “use-by date” when the product remains fresh. Of course, the absence of bacteria may not be the only reason to reject food after a date. Conversely, products that have been exposed to lack of refrigeration and then re-refrigerated may spoil even before their printed expiration date.

Cells of organisms undergo metabolic processes yielding a transfer of electrical charges to and from their membranes to the environment. Therefore, they produce electrical energy during metabolism and reproduction. When these cells are incubated in specialized devices or specialized biochemical fuel cells, the charges generated over time produce a detectable voltage and current. The electrical output from these devices is typically micro-amps with millivolt potentials. Further, an increase in electrical signal output indicates a growth of organisms. Individual or small numbers of pathogens are often harmless because the body can destroy them. However, the uncontrolled replication by mitotic reproduction is often dangerous to the host. Bacteria and other pathogens typically divide mitotically every 15 minutes to 2 hours, increasing their numbers geometrically and multiple times before their death. Thus, the accumulated and summed electrical output is measurable and increases in relatively short periods.

By categorizing the electrical output or “growth” signals for various living substances along with various pathogens in unison and in combination, the background signal from normal metabolic processes can be subtracted from the total signal potential when unwanted pathogens are present and multiplying. Further, by categorizing the magnitude of this electrical growth potential for various pathogens in unison and together, the resulting growth potential can predict quantitatively the presence of the particular pathogen(s) present because the growth increase is different and can be known for different pathogens at different temperatures.

It may be important that the categorized growth rate is also determined relative to the surrounding temperature because temperature affects the pathogen growth rate. Knowledge that only certain pathogens exist in certain environments and temperatures aids the method. Thus, the categorical knowledge from the measured output signals strongly indicates the type or kind of pathogen present by a process of elimination when measuring the growth rate signal at measured temperatures and comparing these signals to the baseline categorical database signals. In this way, it is possible to predict with higher probability the specific pathogen(s) present by subtracting out the baseline signal at the measured temperature and comparing it to the various pathogen databases.

The technique for measuring the electrical signal output from the unknown pathogen or microorganism involves strategically placing the sensor(s) in the area of interest and waiting for the random migratory passage of the pathogens from their present location into the sensor(s). Because of this uncertainty when, or whether, a pathogen will encounter the sensor, choosing the size, placement and number of sensors should increase the probability of detecting any pathogens. Making the sensors of varying sizes also may increase the probability of detection. Therefore, sensors may range in size. Some may cover larger surface areas. This flexibility assists in enhancing the probability of measuring the presence of a pathogen.

Applicants contemplate that sensors could be made as small as the diameter of a surgical needle using nanotechnology. Therefore, one or more may be implanted into human or other living tissue as “real-time” incubators with the ability to measure and or detect the growth of bacteria. Similarly, miniaturizing the sensors could allow an array of sensors to remain small and manageable.

Each bacterium has its own specific growth rate under controlled fuel-cell conditions. Therefore, each produces its own specific voltage versus time growth-plot. Thus, sensors for each microbe could have a different substrate that is advantageous to a specific bacterium. Specific antibiotics act like specific inhibitors to the individual bacteria. Therefore, an array of sensors with different substrates and different inhibitors could differentiate among different bacteria. By knowing the voltage versus growth-time for the most common bacteria, the process can use an algorithm that reviews the outputs from a plurality of sensors and eliminates microbes from sensors that generate no signals or signals below a threshold. Therefore, the method narrows the unknown microbe down to the most probable candidates.

One proposed sensor involves the utilization of two separate cavities—one containing an anode (negative terminal) along with the pathogen and a food source (such as carbohydrates), and one containing a cathode (positive terminal) with moisture (water)—separated by a semipermeable membrane allowing charge transfer across the semipermeable membrane. A single cell device also is contemplated.

The sensors can use inexpensive, disposable and flexible materials. The biosensors may be made by laminating flexible, thin plastic or other sheets together with internal electrodes, anode and cathode, separated by a semipermeable membrane between the outer sheets. However, unlike conventional biochemical fuel cells, the outer, thin sheets surrounding the anode use porous materials to allow pathogens to migrate into the anode to act as the catalyst if pathogens are present to sense the electrical signal occurring as the pathogens metabolize the carbohydrates or glucose fuels seeded in the anode cavity. Similarly, porous materials surrounding the cathode allow the free exchange of gases and oxygen into the cathode chamber.

The porous material could be Gore-Tex®, an expanded and porous polytetrafluoroethylene (ePTFE), or a similar material. Typically, Gore-Tex has about 9 billion pores/in² or around 1.4 billion pores/cm². Thus, the average pore has a length of about 11 μ-in or 27 μ-cm (0.27 μ-m). Typical bacteria have dimensions of about 8 μ-in or 20 μ-cm, so ePTFE could be a suitable option as the outside semipermeable membrane. Vapors can pass across an ePTFE boundary, but liquid water droplets cannot pass across the boundary due to surface tension effects. The top sheet 130 could also be made of a similar porous semipermeable material to allow O₂ exchange from the cathode end.

A signal processor such as a computer or other electronic device can read, analyze and process signals generated by the sensors. Instead of electronic analysis, the output of the sensor can act on disposable electro-photo-sensitive paper, which can change color based on accumulated charge. By “color,” applicants also mean shades of color or gray. The color sensor could be built into the top portion of the sensor itself. With this embodiment, the sensor could be useful for medical bandages or packaging for perishable foods. Using color sensors sensitive to different voltages or signals, one might use an array of voltage sensitive papers sensitive to different voltages to determine which microbe is present.

The system also could amplify the output signals into a recording device for data logging and post-processing. This approach could be useful for real-time data collection in Petri-dish laboratory growth samples or recording of data from air conditioner vents in buildings, hospitals or even residences.

The sensors may be connected electrically parallel or in series. There may be benefit to series connections for amplifying the output signal by summing the signal from multiple sensors.

### BRIEF DESCRIPTION OF THE DRAWINGS

FIG. 1 is a schematic of a conventional biological fuel cell.

FIG. 2 is a side, sectional view of applicants' two-chamber biological fuel cell sensor.

FIG. 3 is a plan view showing an array of applicants' biological fuel cells.

FIG. 4 is a schematic of any array of applicants' biological fuel cells in electrical series.

FIG. 5 is a chart showing the size range for various microbes, other molecules and atoms.

FIG. 6A is a perspective view of an adhesive bandage with applicants' biological fuel cells. FIGS. 6B and 6C shows the bandage applied to the back of a person's hand. In FIG. 6C, the change in color indicates the presence of detected microbes.

FIGS. 7A and 7B are figures of a food package containing meat and applicant's biological fuel cell being exposed to potential contamination from microbes. FIG. 7B shows the food being removed from the package, and a portable device can read the output of the fuel cell.

FIG. 8 is a schematic of a one-chamber biological fuel cell.

When detailed descriptions reference one or more drawing figures, the element being discussed is visible in that drawing. The element also may be visible in other figures. In addition, to avoid crowding of reference numerals, one drawing may not use a particular reference numeral where the same element is in another drawing with the reference numeral.

### DETAILED DESCRIPTION

A microbial fuel cell (biofuel cell) is a biological reactor that converts chemical energy present in the bonds of organic compounds into electric energy through the catalytic reactions of microorganism, typically in aerobic conditions. They use biocatalysts for the conversion of chemical energy to electrical energy.

Microbial fuel cells share similarities with conventional fuel cells, but instead of relying on inorganic catalysts like platinum or other noble metals, they use biocatalysts such as enzymes or whole living organisms as catalysts for converting chemical energy into electricity.

Microbial fuel cells can operate in two ways. They can use biological catalysts—enzymes extracted from biological systems—to oxidize fuel molecules at the anode and to enhance oxygen reduction at the cathode of the fuel cell. Alternatively, whole microbial cells can be used as catalysts in the fuel cells. In both cases, electrical coupling of the biological components of the system with the fuel cell's electrodes must occur. Molecules known as electron-transfer mediators can provide efficient transport of electrons between the biological components, enzymes or microbial cells, and the anode electrodes of the fuel cell. Further, integrated biocatalytic systems that include biocatalysts, electron-transfer mediators and electrodes have been recently developed and utilized in fuel cells. The typical chemical reactions occurring inside the anode and cathode chambers for aerobic bacteria include CₓH_{y}O_{z}+H₂O→CO₂+e⁻+H⁺ (anode) and O₂+4H⁺+4e⁻→2H₂O (cathode).

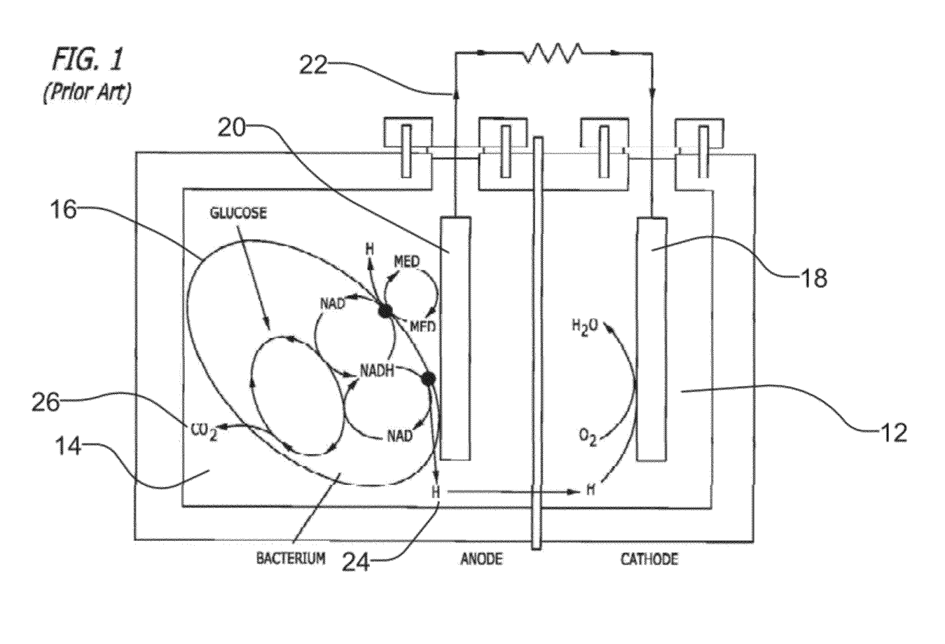

The FIG. 1 schematic shows two chambers, cathode chamber 12 and anode chamber 14. The cathode chamber contains cathode 18 in a liquid medium such as water or hydrogen peroxide. Likewise, the anode chamber contains anode 20 suspended in a liquid medium. Bacterium 16 is suspended in the anode chamber's medium. A conductor 22 electrically connects the anode and cathode. Electronics or other devices (not shown) can read the current or voltage.

Nicotinamide adenine dinucleotide (NAD) is an important cell coenzyme. In a biological redox (oxidation-reduction) reaction, NAD interconverts between reduced nicotinamide adenine dinucleotide (NADH) and oxidized (NAD+) forms. The removal of hydrogen atoms accomplishes most oxidation. For NAD, the reaction is NAD⁺+2H→NADH+H⁺. FIG. 1 shows the reaction schematically including the H⁺ ions 24.

Electron-transfer mediators MED may also be present to assist the reaction. Some microbes are more resistant than others are to the redox reaction and may need mediators to help strip off electrons.

NAD also is involved with glycolysis. Glucose oxidizes to pyruvic acid. NAD⁺ is the oxidizing agent, which reduces to NADH. Pyruvate oxidizes further to CO₂, 26 in FIG. 1.

This is a simplified explanation. Some of it applies only to aerobic metabolism, and the redox reaction may work differently for anaerobic microbes. Nonetheless, the reaction generates ions, which creates an electrical potential between the anode and cathode, which generates current through conductor 22.

The redox reaction varies in different microbes. Though the redox mechanism is similar, the rate of the reactions varies and depends on the cell's metabolism. Therefore, the voltage between the anode and cathode varies depending upon microbe type, temperature and growth time.

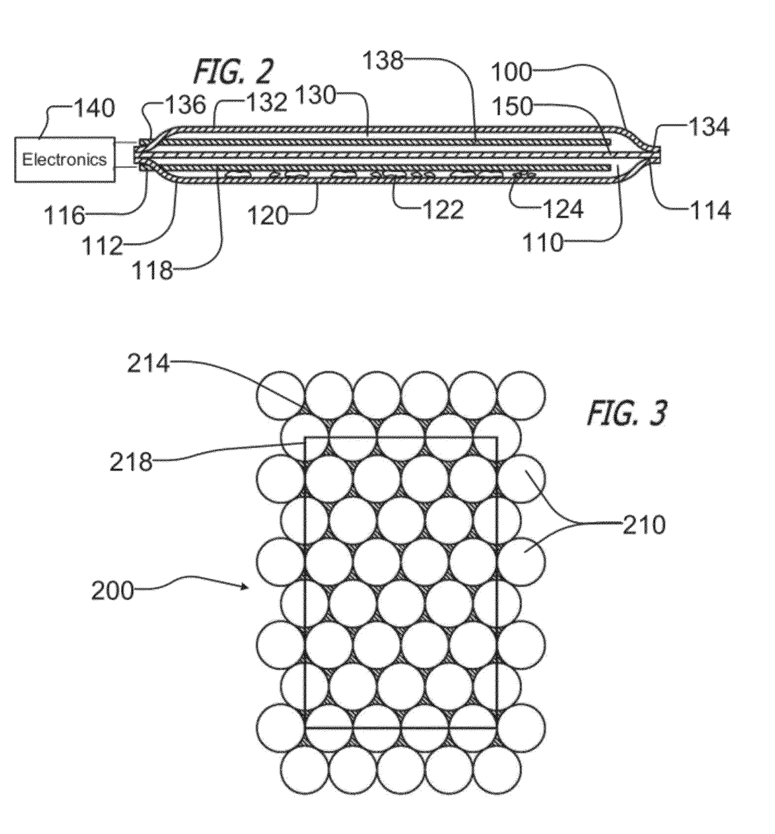

One sensor works by forming two half-cell chambers separated by a semipermeable membrane. The reaction can occur in applicants' sensor such as sensor 100 (FIG. 2). This sensor has two, half-cell chambers, anode chamber 110 and cathode chamber 130. The anode chamber has an outer wall 112 that may be formed of flexible material. Likewise, the cathode chamber includes outer wall 132. Semipermeable membrane 150 separates the anode and cathode chambers. The ends 114 and 116 of the anode chambers outer wall 112, attach to the respective ends 134 and 136 of the cathode chambers outer wall 132. By “attached,” applicants mean that the ends attach directly to each other or that the ends of semipermeable membrane 150 may be intermediate the ends. Those ends may be heat-sealed or otherwise attached to semipermeable membrane 150. As FIG. 2 shows, sensor 100 can be made thin, flexible, inexpensively and disposable, features that may be useful for many applications.

Anode 118 is located in anode chamber 110. It may extend through outer wall 112 as shown in FIG. 2. A wire or other electrical connecter may extend through outer wall 112 and contact the anode. The anode is shown planar, but it can curve, especially near outer wall end 116. Similarly, cathode 138 in cathode chamber 130 may extend through outer wall 132 and may attached to a wire or other electrical connecter through outer wall 132 to contact the cathode. The cathode also can curve, especially near outer wall end 136. The anode and cathode connect to signal processor 140, which may be a computer, special electronics or another device for indicating current or voltage between the anode and cathode.

When choosing the type and kind of material for anode 118 and cathode 138, satisfactory attributes may include good conductivity, acceptable physical strength, high surface area, favorable surface properties, good chemical and electrochemical stability and low cost. Graphite fiber cloth, graphite fiber felt and carbon fiber paper are candidates for the materials. Alternatively, a graphite powder could be sprayed or brushed onto a semipermeable membrane for the anode or cathode. Permanent anodes and cathodes used in biochemical fuel cells, which are designed for generating electricity are usually designed to be more permanent. Therefore, their design must account for longer-term use. Applicants' anodes and cathodes are in disposable devices that may operate for a few days more or less. Thus, cost will be important. Nevertheless, the anodes and cathodes cannot deteriorate or break before the sensor must be used. Having the outer anode and cathode surfaces made of a semipermeable material probably is important to the use and operation of the biosensor because pathogens need access paths into the sensor on the anode side and oxygen and gas exchange on the cathode side.

In addition, voltage output is a function of the surface area of the anode and cathode. Because the sensors can be made small, and the output voltage is very small, the anode and cathode has a relatively large surface area in the small space of the sensors' chambers 110 and 130.

At least a portion 120 of outer wall 120 of the anode chamber 110 is a porous semipermeable membrane having pores large enough for ingress and egress of those microbes of interest such as bacteria 122. FIG. 5 shows the sizes of various microbes. Knowing the sizes is a useful guide for pore sizes. Bacteria are prokaryotes because they contain no cell nucleus, or any other membrane-bound organelles.

The semipermeable membrane may be Gore-Tex® or similar material. Using woven fabric treated with a hydrophilic substance on the outer surface and a hydrophobic substance on the inside surface is another possibility. The substances hold liquid within chamber 110. Anode chamber may contain fluid passed through the semipermeable membrane, or it may contain fluid before use. Membrane 120 restrains fluid within the anode chamber. Anode chamber 110 also may contain glucose or carbohydrate 124 to feed the microbes' metabolism.

Part of outer wall 132 of cathode chamber 130 also could have a porous semipermeable membrane.

Cathode chamber also contains liquid, preferably water or hydrogen peroxide, H₂O₂. Aerobes, organisms that require oxygen or that live in the presence of oxygen, produce hydrogen peroxide naturally as a by-product of metabolism. Aerobes have enzymes that can decompose low concentrations of H₂O₂ to water H₂O and oxygen O₂.

Semipermeable membrane 150 is a proton exchange membrane. They usually are made from ionomers. They conduct protons but do not allow gases to permeate. DuPont's Nafion is a commonly used proton exchange membrane. Gelatinous agar with salt also acts as a proton exchange membrane. Its low cost may make it suitable for applicants' sensor.

As the microbe metabolizes and reproduces, H⁺ ions are generated. That generates an electrical potential between anode 118 and cathode 138, which causes a current to flow from the anode to the cathode.

The current probably will be very small, in the order of micro-amps. The potential likely will be in the millivolts. Nonetheless, sensitive instruments can read the electrical signal. Instead of using electronic instruments, it may be possible to sense the generated electricity with color-changing paper or other chemical sensitive to current or voltage.

Standard biochemical fuel cells require adding glucose or other carbohydrates to continue the redox reaction. Applicants can rely on new microbes from outside the device to pass the semipermeable membrane 122 into the anode chamber 110. Also, in the disposable version of the proposed biochemical sensors, applicants are proposing using the sensors for a relatively short active period (on the order of days) and so no new fuel source (carbohydrate) is needed to be loaded within the sensor.

The sensors can be very small. That creates a potential issue. If one sensor or a small array of sensors is placed on a wound, a portion of a wound could be badly infected while the area under the sensor could be relatively free of microbes. Similarly, one area could be infected with one pathogen, but the region near the sensor could have a different pathogen. The different pathogens might have dissimilar reactions to antibiotics a physician might prescribe.

Therefore, mounting separate sensors in a tightly packed array that covers the entire region of interest could minimize that problem. FIG. 3 shows such an array 200 of sensors 210. The sensors can be held together with adhesive. In FIG. 3, the array is on tissue 214. The drawing shows 66 sensors in the array. Applicants expect that the array could have many more sensors. The larger array could be cut into smaller arrays. Cutting could damage sensors along the cut, but enough sensors will remain to provide sufficient readings. The electronics could ignore any sensors along the periphery of the array. Alternatively, a large array could have spaces between some sensors so that cuts of larger arrays into smaller ones would be along the spaces.

Cover 218 (FIG. 3) can connect electrically to each sensor, or the connection can be elsewhere. The electrical output can be a sum of all the signals generated by the sensors 210. The electronics also could provide a readout for each sensor or for a group of adjacent sensors. For example, the sensors could be numbered 1, 2, 3 . . . for the first row along the top in FIG. 3. The next row could be 7, 8, 9 . . . . The various sensors can be linked. For example, the signal from adjacent sensors 1, 2, 3, 7, 8 and 9 could be combined, and the signals from other groups of sensors also could be combined separately. Combining the output of six sensors is an example and may not be ideal. Using an array of adjacent sensor may help to locate an infected area.

Alternatively, signals from non-adjacent sensors could be combined. If any group shows signals indicating infection, the physician would start an antibiotic regimen for the identified infection.

If the electronics monitor groups of more than one sensor, the sensors may be connected in parallel or in series. Connecting them is series is probably preferable because the tiny voltages from each will add. Connecting the sensors in parallel yields more current, but a parallel connection probably is less desirable because the individual sensors can have mismatched voltages. However, the process used to manufacture the sensors should insure that no sensor is an open circuit. Even if a few sensors become open circuits, applicants believe that with sufficient sensors, the system can have enough redundancy to account for an occasional open circuit.

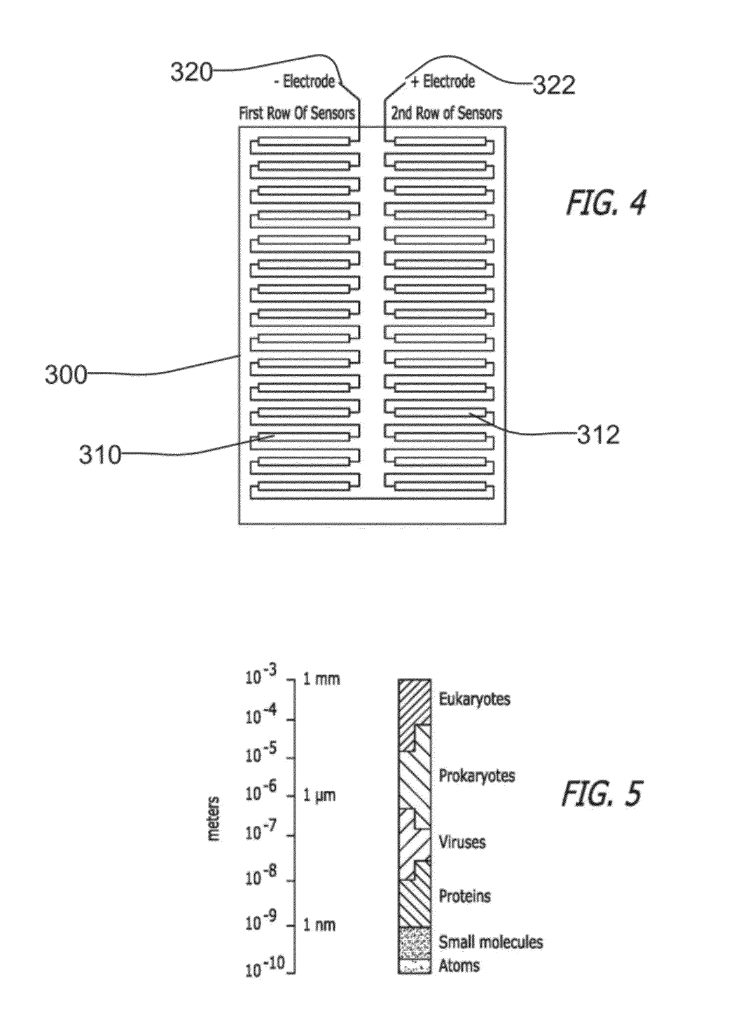

FIG. 4 shows a schematic of an array 300 of sensors in two rows. Sensors 310 are in one row, and sensors 312 or in the second row. In this arrangement, the voltage between the negative electrode 320 and the positive electrode is the sum of the voltages from all the sensors in array 300.

In FIG. 3, the sensors are round (shown as circular). An oblong shape may be desirable. Triangular, rectangular, hexagonal and other shapes can pack more tightly, but they have corners in which the reaction may not work as well. Therefore, circular or other rounded shapes may prove beneficial.

The two outer material sheets may be fabricated from a semi-porous material to allow the egress of pathogens to pass into the anode chamber (where the carbohydrate food source is located) but not allow fluids to pass freely through the device walls.

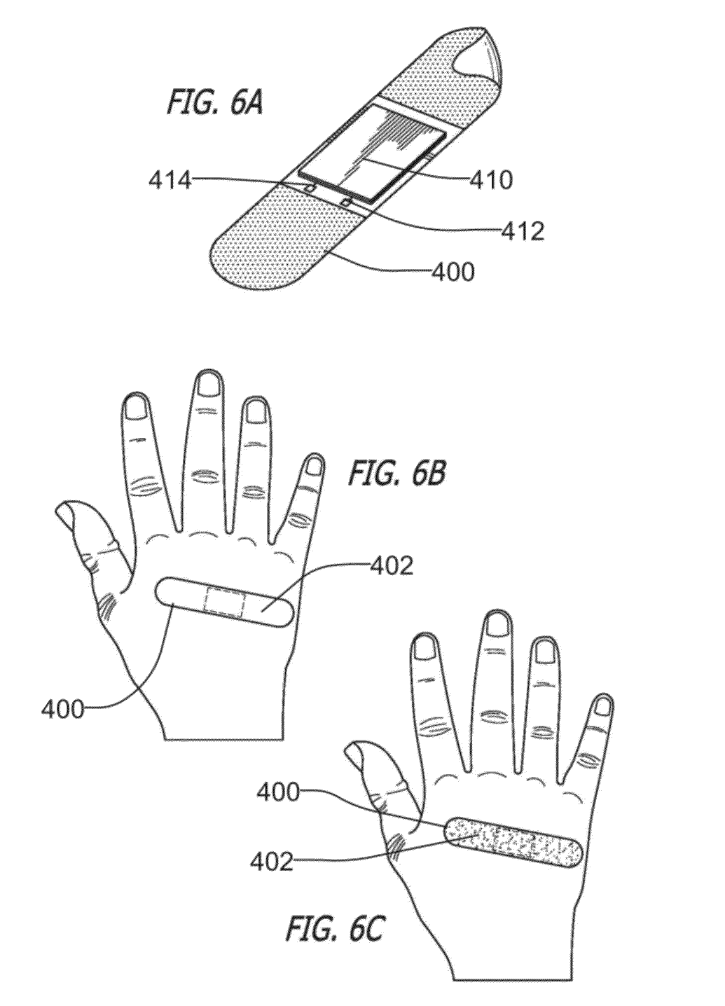

The sensor or array of sensors could be mounted in or on a bandage. FIG. 6A shows an array of sensors 410 mounted on an otherwise conventional adhesive bandage 400. The sensors in the array connect electrically, and the signal can be read at pads 412 and 414. The pads are shown on the inside of the bandage, but the bandage material would have openings (not shown) on the top side.

FIG. 6B shows such a bandage 400 on the back of a hand. Instead of using electronics to read a signal from the sensor array, applicants contemplate that electrical activity could change the color of the top surface 402 of the bandage. See FIG. 6C.

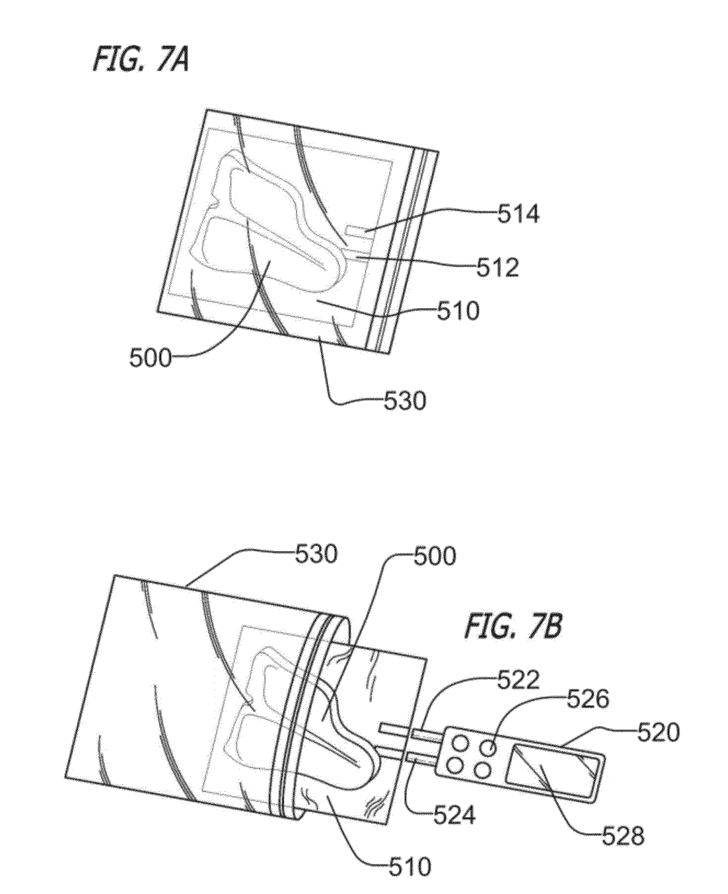

FIG. 7 shows the use of the sensor to monitor food spoilage using applicants' sensor. Meat, for example steak 500, is contained within a top and bottom sheet of material. Only a top sheet 510 is visible. A single sheet also may be satisfactory. The steak and sheet are within sealable container 530.

Scattered on the inside of the sheet(s) are sufficient numbers of applicants' sensors (not shown) to contact a sufficient portion the meat to detect spoilage. A steak is not homogeneous. It contains muscle meat, bone, fat and connective tissue. Ground meat is more homogeneous than steak, but a package may contain meat from different animals or from different parts of the same animal. Packaged chicken contains different animal parts such as skin. A package of fish may contain filets from different fish. Whole fish has a head, tail, fins, etc. Further, the size of the food may vary relative to the size of sheet 510. Therefore, the number of sensors may vary depending on the food. Conversely, the sheet could have enough sensors to be in contact with enough areas of the largest food.

Though the discussion focused on meat, poultry and fish, the system could extend to vegetables, dairy and other food. Sensors for dairy or other packaged food could be built into the container. They also could be attached to the packaging for fresh vegetables. Containers for home food storage could mount to the container.

As with previous uses for the sensors, the food could be monitored constantly. That is, the sensors could be electronically connected to electronic monitors. They also could use color change material. Continuous monitoring of hundreds of food packages in a supermarket likely is impractical. Instead of wired connections, the sensors could connect to an RFID chip that could transmit the sensor signals to a signal processor such as a computer or other local monitor.

RFID chips could also be used to monitor mold sensors in regions of buildings by relaying measured signals from the mold sensors short distances back to a nearby staged control center. The control center may relay the aggregate room data back to a master control station for an entire building or group of buildings. If unknown water damage produces mold growth within a room or rooms of a home or commercial building, such an arrangement could pinpoint the infected location for quicker remediation and removal.

Another process uses a separate reader 520. Film 510 has two electrodes 512 and 514. In a sense, when the sensors first contact the food, the sensors act like an uncharged battery because they lack microbes or pathogens within them that have passed into the anode chamber to “activate” the sensor and begin the metabolic action inside the anode chamber. When the microbial catalysts activate the sensor, the sensor takes time to “charge” to a measurable voltage. Reader 520 has two probes 522 and 524 that can contact the electrodes on the film. The reader may have on-off and function buttons 526. FIG. 7B. Function buttons allow for selecting the type of food being monitored based upon the signal strengths from the types of bacteria that predominate on different foods. For example, E-Coli is a more common contaminate in meats, raw milk, uncooked fruits and vegetables, and Salmonella is less prevalent in vegetables but still found in meats and dairy products. Screen 528 on the reader has a display for showing what microbe is monitored. The presence of metabolic activity also is displayed.

Reader 520 is a small electrical device that reads the electrical voltage output from the sensor films. The reader allows quick reads after microbes in the sensors have had time to incubate and generate an electrical potential. One could connect the reader temporarily connected to the output electrodes 512 and 514 to read any metabolic signal. The reader also could be clipped onto the packaging so that electrodes 512 and 514 remain connected to the reader's electrodes 522 and 524.

Although this arrangement is discussed for food monitoring, it also could be used for monitoring wounds, other medical applications and for mold detection.

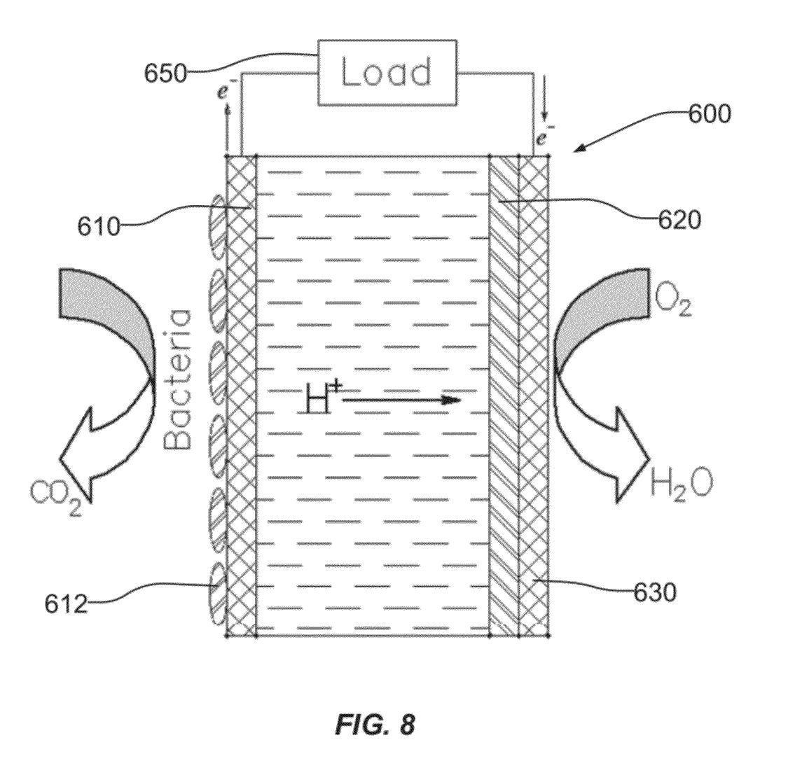

FIG. 2 showed a dual chamber microbial fuel cell. FIG. 8 shows the construction of a single chamber microbial fuel cell 600. Anode 610 and cathode 630 may be made of conductive, semipermeable material. For example they may be carbon fiber cloth coated with graphite powder spray to facilitate the conduction of current. Insofar as the cloth might allow liquids to flow through them, the cloth may be coated to prevent the flow. The anode could also be coated with a carbohydrate substance as a fuel source for the microbes if bacteria 612 are present. Protein exchange membrane 620 may be a coating or film of salt and agar. The spacing between the anode and cathode may vary. It is exaggerated in FIG. 8.

Metabolizing and reproducing microbes generate H⁺ ions, which produces an electrical potential between anode 610 and cathode 630. The protons, H⁺ ions, pass through the cathode and react with O₂ from the air to form H₂O. The reaction generates a potential between the anode and cathode. Thus, current flows between the anode and cathode though load 650. The semipermeable membranes forming the anode and cathode prevent water and air from passing from the outside into the microbial fuel cell. Although not shown in FIG. 8, a tougher material may cover the outside of the anode and cathode to protect them. This tougher material also would be semipermeable to allow bacteria to reach the anode and the water created by the redox reaction to exit through the cathode.

A single chamber biosensor can be thinner, may produce a larger output signal for the same surface area sensor due to lower internal resistance, and may cost less.

The description is illustrative and not limiting and is by way of example only. Although this application shows and describes examples, those having ordinary skill in the art will find it apparent that changes, modifications or alterations may be made. Many of the examples involve specific combinations of method acts or system elements, but those acts and those elements may be combined in other ways to accomplish the same objectives. With regard to flowcharts, additional and fewer steps may be taken, and the steps as shown may be combined or further refined to achieve the methods described. Acts, elements and features discussed only in connection with one embodiment are not intended to be excluded from a similar role in other embodiments.

“Plurality” means two or more. A “set” of items may include one or more of such items. The terms “comprising,” “including,” “carrying,” “having,” “containing,” “involving,” and the like in the written description or the claims are open-ended, i.e., each means, “including but not limited to.” Only the transitional phrases “consisting of” and “consisting essentially of” are closed or semi-closed transitional phrases with respect to claims. The ordinal terms such as “first,” “second,” “third,” etc., in the claims to modify a claim element do not by themselves connote any priority, precedence, or order of one claim element over another or the temporal order in which acts of a method are performed. Instead, they are used merely as labels to distinguish one claim element having a certain name from another element having a same name (but for use of the ordinal term). Alternatives such as “or” include any combination of the listed items.

We claim:

1. A sensor for determining the presence of microbes comprising:
a. a first chamber formed of at least one outside wall, the first chamber containing a food source for microbes,
b. an anode in the first chamber capable of being connected to a first electrical terminal,
c. at least a portion of the outside wall of the first chamber comprising a semipermeable membrane through which microbes can pass into the first chamber,
d. a second chamber formed of at least one outside wall, the second chamber containing a cathode capable of being connected to a second electrical terminal, and
e. a membrane between the first and second chambers capable of allowing ions to pass between the chambers.

2. The sensor of claim 1, wherein the anode is semipermeable.

3. The sensor of claim 1, further comprising an electrical connection between the anode and the cathode.

4. The sensor of claim 3, further comprising means for reading the signal in the electrical connection.

5. The sensor of claim 3, further comprising a signal processor in the electrical connection.

6. The sensor of claim 1, wherein the outside wall of the first chamber is flexible.

7. The sensor of claim 1 wherein the outside wall of the first chamber and the outside wall of the second chamber are attached to each other.

8. The sensor of claim 1, further comprising carbohydrate in the first chamber.

9. The sensor of claim 1, further comprising an inhibitor in the first chamber for inhibiting the growth of particular microbes.

10. A device for determining the presence of microbes comprising:
a. a sensor comprising a microbial fuel cell comprising an anode and a cathode,
b. a semipermeable membrane constructed to allow microbes to pass into the sensor, the sensor generating an electrical signal when microbes pass into the sensor and metabolize,
c. a signal processor capable of being electrically connected to the anode and the cathode, the signal processor reading and analyzing the signal.

11. The device of claim 10, wherein the signal processor comprises material that changes color when exposed to an electrical signal.

12. The device of claim 10, wherein the sensor comprises:
i. a first chamber formed of at least one outside wall, the first chamber containing a food source for microbes,
ii. an anode in the first chamber capable of being connected to a first electrical terminal, the semipermeable membrane extending at least along a portion of the outside wall of the first chamber,
iii. a second chamber formed of at least one outside wall, the second chamber containing a cathode capable of being connected to a second electrical terminal, and
iv. a membrane between the first and second chambers capable of allowing ions to pass between the chambers.

13. The device of claim 10, further comprising multiple sensors arranged in an array.

14. The device of claim 10, further comprising exposed electrodes electrically connected to the sensor, the electrodes being positioned such that they can be temporarily contacted to the signal processor.

15. The device of claim 10 wherein the sensor comprises:
i. an anode formed of a conductive semipermeable material allowing the passage of microbes through the material,
ii. a cathode, and
iii. a membrane between the anode and cathode for passing ions generated by the anode to pass to the cathode.

16. A process for determining the presence of microbes comprising:
exposing a closed microbial fuel cell to the environment,
allowing microbes in the environment to pass into the closed fuel cell through a semipermeable membrane, and
analyzing any electrical signal generated by the microbes metabolizing in microbial the fuel cell, the signal indicating the presence of microbes passing into the closed fuel cell through a semipermeable membrane.

17. The process of claim 16, further comprising mounting the microbial fuel cell adjacent a wound.

18. The process of claim 16, further comprising mounting the microbial fuel cell adjacent food

19. The process of claim 16, further comprising mounting the microbial fuel cell against building structure.
